(19) United States
(12) Patent Application Publication
(10) Pub. No.: US 2018/0235209 A1
(43) Pub. Date: Aug. 23, 2018

(54) Pest Protective Apparatus For Controlling Plant Pathogens

(71) Applicant: Luxembourg Institute Of Science and Technology (LIST), Esch/Alzette (LU)

(72) Inventor: Bruno PRINTZ, Escherange (FR)

(21) Appl. No.: 15/750,979

(22) PCT Filed: Aug. 3, 2016
(86) PCT No.: PCT/EP2016/068512

§ 371 Date: Feb. 7, 2018

(30) Foreign Application Priority Data
Aug. 7, 2015 (LU) LU92794

(51) Int. Cl. A01N 25/08; A01N 25/04; C12N 11/14

(52) U.S. Cl. CPC A01N 25/08; C12Y 302/01147; C12N 11/14; A01N 25/04

(57) ABSTRACT

A pest protective apparatus adapted for controlling plant pathogens. The pest protective apparatus comprises at least one fibrous network and/or at least one fibrous cover. The pest protective apparatus is remarkable in that the at least one fibrous network and/or the at least one fibrous cover comprises a system in which at least one precursor is transformed into a biopesticide and/or repellent product.

### CROSS-REFERENCE TO RELATED APPLICATIONS

The present invention is the US national stage under 35 U.S.C. §371 of International Application No. PCT/EP2016/068512, which was filed on Aug. 3, 2016, and which claims the priority of application LU 92794 filed on Aug. 7, 2015, the content of which (text, drawings and claims) are incorporated here by reference in its entirety.

### FIELD

The invention is directed to a pest protective apparatus comprising a system which allows the transformation of a substrate into a biopesticide and/or repellent product which will subsequently control plant pathogens.

### BACKGROUND

Pest management is currently largely performed with synthetic chemicals, but the development of alternative and eco-friendly methods to control pests are encouraged.

Patent application published US 2012/0079625 A1 relates to a method for protecting living plants from harmful insects via a sheet-like structure impregnated with an insecticide which is from synthetic origin.

The field of the biocontrol includes various techniques of integrated pest management such as macro-organisms, micro-organisms, pheromones, kairomones and natural substances originating from animals, minerals and plants to control pest and pathogens. In 2014, Sahayaraj K. summarized the current use of the nanotechnologies in the domain of the plant protection. The few data available on this topic brings to the conclusion that only few research all over the world are currently working with this emerging problematic although there is an urgent need of modern approaches of pest management (Sahayaraj K., Adv. Plant Biopest. (chapter 14), 2014, 279-293).

Substances from various plant families have been reported to exert a repelling activity against fungus, bacteria, nematodes or insects and can be successfully used in the treatment/control of pests (Murthy N. B. K., et al., Indian J. Exp. Biol., 1974, 12, 208-209 and Regnault-Roger C., et. al, Biopesticides d'origine végétale (2ème edition), 2008.) In particular, sulphur containing molecules such as those produced among other by the families Brassicaceae and members of the genus Allium which are enzymatically produced from non-toxic precursors are toxic at low level for a wide range of organisms (Ahuja I., et al, Agron. Sustain. Dev., 2010, 30, 311-348; Auger J., et al, Ecologie, 1994, 93-101; Cutler H. G., et al., Biologically Active Natural Products: Agrochemicals, 1999).

The mixing of biopesticide precursor with active enzymes in liquid form to produce repellent is known to the art and has been proposed for the production of glucosinolate products such as nitrile, thiocyanate or isothiocyanate, and/or mixture (WO 2015/013808 A1). Also known in the art is the use of a two-part pesticide precursor system comprising a glucosinolate concentrate on one side and an active myrosinase complex on the other side both in dry form. When both components are mixed with water, the glucosinolate breakdown products are released to control pests (US 2015/0005172 A1). However, the lifespan of active enzymes in the environment is relatively low, which considerably hinders the reusability of the enzymes. In addition, the substrates are commonly hydrophilic, making them easily leachable and not accessible to the enzymes.

Enhancing the lifespan of enzymes has been successfully performed in the domains of the biocatalysis, biofuels, enzyme-controlled drug delivery and the biosensors by immobilizing the enzymes into silica mesoporous material (Carlsson N., et al., Adv. Colloid Interface Sci., 2013, 205, 339-360; Popat A., et al., Nanoscale, 2011, 3, 2801-2818; Wang Y., et al., Chem. Mater., 2005, 17 953-961). Mesoporous material relates to silica-based material with a pore diameter sufficient to allow the penetration of the enzyme. It can be in the form of SBA-15 (Santa Barbara-15), SBA-16, MCM-41 (Mobil Crystalline Materials-41) or all other type of mesoporous silica sieves/spheres/cages (FSM-16 (Folded Sheet Mesoporous-16), MCM-48, FDU-12 (Fudan University-12), MCF (Mesostructured Cellular Foam), SMS (Sponge Mesoporous Silica), mesoporous carbon, PMOs (Periodic Mesoporous Organosilica), Meso-MOFs (meso-Metal Organic Frameworks) (Zhou Z., et al., Top Catal., 2012, 55, 1081-1100). The pore structure may be folded-sheet, 2D hexagonal channels, cubic, spherical cages, mesocellular foam, sponge-like mesoporous silica, 3D cubic cages. The synthesis of the mesoporous silica is preferentially done in acidic conditions and with the triblock copolymer (Pluronic®P123) as template and tetraethyl orthosilicate (TEOS) as silica source. However, the use of other templates such as among other the triblock copolymer (Pluronic®F127), CTMA (cetyltrimethylammonium) (CₙH₂ₙ₊₁(CH₃)N+, n=8-18), CTAB (cetyltrimethylammonium bromide) and other silica source including among other (3-aminopropyl)trimethoxysilane (APTMOS), sodium silicate or tetramethyl orthosilicate (TMOS) may also give interesting properties to the silica mesoporous material (Zhou Z., et al., Top Catal., 2012, 55, 1081-1100).

Enzyme immobilization into the silica matrix can be carried out through various techniques including cross-linking methods, covalent binding, physical adsorption, encapsulation and entrapment (CN 101451133 B). Carboxylethyl or aminopropyl functionalization of the mesoporous silica also appears as a good technique for improving the catalytic activities of the silica-based biocomposite (Lei C., et al., J. Am. Chem. Soc., 2002, 124, 11242-11243). Functionalization of the silica-material can further be used for covalent linking of the enzyme on the derivatized-silica.

The synthesis of hydrophilic gels can result from the gelation of various natural compounds such as agarose, agar-agar, alginate, pectin, starch or gelatine taken alone or in mixes. They allow the diffusion of small hydrophilic molecules and prevent the diffusion of large particles such as silica mesoporous materials that retain blocked in the polymeric network. Microencapsulation of silica in alginate had remarkable advantages of sustained-release of compounds and stability under different pH values, different temperatures, and UV irradiation. The presence of the hydrophilic gel can further increase the stability of the material upon ageing and limit enzyme leaching (Coradin T., et al, Comptes Rendus Chim., 2003, 6, 147-152). Biocomposite incorporation in hydrophilic gels such as alginate showed much better performance due to the more homogeneous distribution of silica particles in the composite material (Xu S. et al., Ind. Eng. Chem. Res., 2006, 45, 511-517).

The idea of the present invention is to merge all these parts (conception of a structure, using a precursor with active enzymes in liquid form to produce biopesticide and/or repellent product, enhancing the lifespan of said enzymes, use of hydrophilic gels) to produce for the first time a pest protective apparatus which is able to deliver in situ a constant flux of biopesticide and/or repellent product.

### SUMMARY

The invention has for technical problem to provide a pest protective apparatus which is producing a biopesticide and/or repellent product from a precursor which is harmless and easy to handle.

The first object of the present invention is a pest protective apparatus, for controlling plant pathogens, comprising at least one fibrous network and/or at least one fibrous cover. The pest protective apparatus is remarkable in that the at least one fibrous network and/or the at least one fibrous cover comprises a system in which at least one precursor is transformed into a biopesticide and/or repellent product.

In various embodiments, the system is composed of fibres adapted to control plant pathogens which are coated with at least one preparation able to produce biopesticide and/or repellent for controlling plant pathogens.

In various embodiments, the preparation able to produce biopesticide and/or repellent for controlling plant pathogens comprised at least one active enzyme.

In various embodiments, the at least one active enzyme is immobilized within one nanoporous material.

In various embodiments, the one nanoporous material is a mesoporous material.

In various embodiments, the nanoporous material is embedded within one hydrophilic gel.

In various embodiments, the hydrophilic gel is made of agarose, agar-agar, alginate, pectin, starch and/or gelatine, in various instances made of alginate.

In various embodiments, the at least one active enzyme is one active enzyme selected from the group of glycosidase, lyase and/or lachrymatory-factor synthase, in various instances from the group of glycosidase.

In various embodiments, the at least one enzyme from the group of glycosidase is thioglucosidase.

In various embodiments, the at least one active enzyme immobilized within the nanoporous material is covalently bound to the nanoporous material, physically adsorbed to the nanoporous material, encapsulated within the nanoporous material or entrapped within the nanoporous material.

In various embodiments, the at least one active enzyme immobilized within the nanoporous material is cross-linked with glutaraldehyde or with 1-ethyl-3-(3-dimethylaminopropyl)carbodiimide and N-hydroxysuccinimide.

In various embodiments, the nanoporous material is a silica-based mesoporous material.

In various embodiments, the silica-based mesoporous material comprises tetraethyl orthosilicate and/or tetramethyl orthosilicate and/or methyltrimethoxysilane and/or (3-aminopropyl)trimethoxysilane.

In various embodiments, the preparation has a pH comprised between 4 and 11.

In various embodiments, the preparation further comprises the at least one precursor, at least one mineral and/or at least one cofactor.

In various embodiments, the preparation is adapted to receive the at least one precursor, at least one mineral and/or at least one cofactor.

In various embodiments, the at least one mineral and/or the at least one cofactor is phosphate buffer or ascorbic acid.

In various embodiments, the phosphate buffer is at a concentration comprised between 10 mM and 500 mM, in various instances at a concentration comprised between 50 mM and 250 mM, for example at a concentration comprised between 75 mM and 150 mM, e.g., at a concentration of 100 mM.

In various embodiments, the ascorbic acid is at a concentration comprised between 50 μM and 1500 μM, in various instances at a concentration comprised between 150 μM and 1000 μM, for example at a concentration comprised between 250 μM and 750 μM, e.g., at a concentration of 500 μM.

In various embodiments, the pest protective apparatus further comprises means for distributing water to the fibrous network and/or fibrous cover, the means in various instances comprising a cartridge configured for being in fluid connection with the at least one fibrous network and/or the at least one fibrous cover and for being fed with water.

In various embodiments, the cartridge comprises at least one precursor which is transformed into a biopesticide and/or repellent product into the system of the at least one fibrous network and/or the at least one fibrous cover.

In various embodiments, the cartridge further comprises at least one mineral and/or at least one cofactor.

It is a second object of the present invention to disclose a method for controlling plant pathogens with a pest protective apparatus. The method is remarkable in that the pest protective apparatus is in accordance with the first object of the present invention and in that the method comprises the first step of activating at least one precursor by addition of water to the system of the at least one fibrous network and/or the at least one fibrous cover to produce a biopesticide and/or repellent product for controlling plant pathogens; and the second step of applying the biopesticide and/or repellent product resulting from the first step to plants, in various instances vegetables and/or fruits, in order to prevent pest attacks. The first step of activating at least one precursor can also be, for example, the catalytic splitting of the at least one precursor.

In various embodiments, the at least one precursor is comprised in the pest protective apparatus, the pest protective apparatus in various instances also comprising at least one mineral and/or at least one cofactor.

In various embodiments, the at least one precursor is added to the pest protective apparatus, the pest protective apparatus in various instances comprising at least one mineral and/or at least one cofactor.

In various embodiments, the at least one precursor is added with also at least one mineral and/or at least one cofactor to the pest protective apparatus.

The invention is particularly interesting in that the precursor of the biopesticide and/or repellent product which is provided by the pest protective apparatus of the invention is chosen among the inactive and non-toxic forms of the potential organic derivatives. The biopesticide and/or repellent preparation which is coated onto the fibres of the fibrous network and/or fibrous cover further comprises immobilized enzyme which provides enhanced enzymatic activity compared to a non-immobilized enzyme.

### DRAWINGS

FIG. 1 illustrates a pest protective apparatus in accordance with various embodiments of the invention.

### DETAILED DESCRIPTION

The present invention relates to a pest management biocontrol system made of active biocatalysts able to produce natural repellents and/or biopesticides by means of controlled enzymatic reactions.

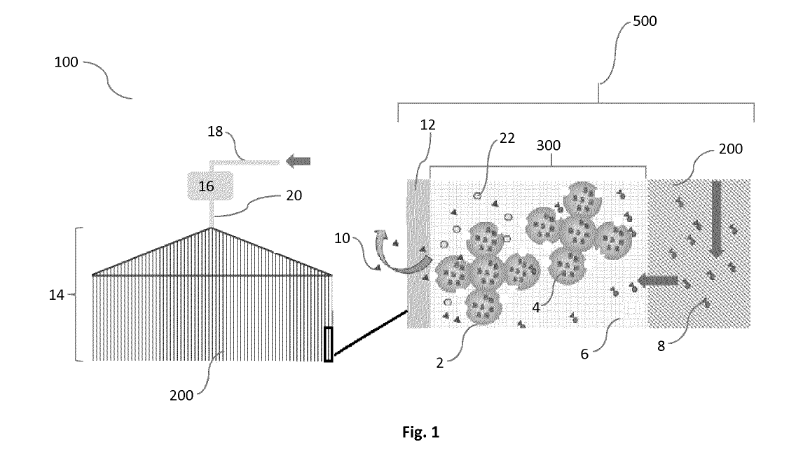

As depicted on FIG. 1, the pest protective apparatus 100 of the present invention comprises at least one fibrous network and/or at least one fibrous cover 14.

The network and/or the cover 14 comprises a system 500 in which at least one precursor 8 is transformed into a biopesticide and/or repellent product 10.

The system 500 is made of fibres 200 which are in fact coated with a preparation 300 which presents biopesticide and/or repellent properties. Such type of preparation comprises at least one active enzyme 4 which is capable to transform at least one substrate 8 into a biopesticide and/or repellent product 10, which will subsequently control plant pathogens.

In order to enhance the lifespan of the at least one enzyme 4, the biocatalyst is immobilized within one nanoporous material 2, in various instances into a mesoporous silica matrix, more particularly into a silica-based mesoporous material, the whole being glued or embedded into a hydrophilic gel 6.

The mesoporous materials of the present invention are ordered silica-based mesoporous particles with a narrow pore size distribution, a well-defined pore geometry and a well-defined pore connectivity.

The pore size distribution and the global geometry of the mesoporous material are defined according to the properties of the biopesticide-producing enzymes and/or repellent-producing enzymes that are immobilized into the mesoporous structure.

The pore size of the silica-based mesoporous material is comprised between 5 nm and 30 nm.

The immobilization procedure should allow to optimize the catalytic activities of the enzymes in comparison with the free enzymes and to enhance their reusability in order to produce a constant and sufficient flux of biopesticide and/or repellent product allowing a sufficient biopesticide and/or repellent activity against the targeted pathogens.

The silica-based mesoporous material will be synthesized in acidic conditions using a silica precursor that could be among other tetraethyl orthosilicate (TEOS), and/or tetramethyl orthosilicate (TMOS), and/or methyltrimethoxysilane (MTMOS), and/or (3-aminopropyl)triemthoxysilane (APTMOS), in combination with tri-block copolymer mixtures such as Pluronic®F127 and/or Pluronic®F123 as structure-directing agents.

The mesoporous structure can be produced hydrothermally and/or by sol-gel synthesis and can be functionalized.

The enzymes that can be immobilized into the mesoporous material are those involved in the natural responses of the plants when facing a pathogen attack. The biopesticide and/or repellent compounds produced during the enzymatic reaction can be a bactericide, a fungicide, an insecticide and/or a nematicide.

The enzyme from the glycosidases, oxidoreductases, the transferases, the hydrolases, the lyases, the isomerases and the ligases can be immobilized into the mesoporous material.

Preferred transferases are those belonging to the glycosidases class, i.e. the enzymes hydrolysing O-glycosyl and S-glycosyl compounds.

Preferred lyases are, for example, carbon-sulfur lyases, in particular alliin lyase (also known as alliinase).

Enzymes from the PF10604 family, i.e. the lachrymatory-factor synthase, can also be used in the present invention.

A preferred glycosidase is thioglucosidase. This is the enzyme of choice which has been tested for transforming a precursor into a biopesticide and/or repellent product.

The immobilization procedure can be performed through the following techniques known in the art: covalent binding, physical adsorption, encapsulation and entrapment.

The immobilization includes the penetration of the enzymes inside the mesoporous material, through the nanopores and an eventual step of cross-linkage between the enzymes using gluturaldehyde or 1-ethyl-3-(3-dimethylaminopropyl)carbodiimide and N-hydroxysuccinimide.

The mesoporous biocatalyst will then be embedded in a hydrophilic gel 6 made of agarose, agar-agar, alginate, pectin, starch, gelatine taken alone or in mixes, in various instances alginate.

The use of alginate allows a gelation without heating. Only CaCl₂ must be added for the gelation step.

The hydrophilic gel allows the diffusion of the products of the enzymatic reaction, the diffusion of the substrates of the enzymes and the confinement of all chemicals required to maintain the enzymes in a working status.

The hydrophilic gel 6 can contain all minerals and cofactors indispensable for the enzymatic activities. This can be a phosphate buffer (pH=6.1) at a concentration comprised between 10 mM and 500 mM, in various instances at a concentration of 100 mM and/or ascorbic acid at a concentration comprised between 50 μM and 1500 μM, in various instances at a concentration of 500 μM.

In case the minerals and cofactors are not added before the gelation step, they can be added by diffusion within the gel by soaking into an aqueous buffer comprising these compounds.

The pH of preparation 300 able to produce a biopesticide and/or a repellent for controlling plant pathogens is in various instances comprised between 4 and 11. In particular, the pH of the hydrophilic gel allows an optimal activity of the immobilized enzymes.

Upon addition of the substrate 8 for the enzyme 4 and upon addition of water, and, upon addition of minerals and cofactors for activating the enzyme if they are not formerly included within the hydrophilic gel 6, the enzyme 4 will proceed to the synthesis of a product 10 which will present biopesticide and/or repellent properties. This product 10 will be released and will act to eliminate (in case of biopesticide activity) and/or to keep away (in case of repellent activity) the pest and/or the plant pathogens from the living plants, such as bacteria, fungi, insects, bugs, and/or nematodes.

The scheme on FIG. 1 represents in particular a precursor 8 composed of two chemical moieties, one being the biopesticide and/or the repellent product 10, the other being a compound 22.

In various instances, the product 10 of the enzymatic reaction is a volatile organic compound.

The fibres 200 of the pest protective apparatus 100, which are coated with the preparation 300 able to produce biopesticide and/or repellent for controlling plant pathogens, are in various instances natural fibres. Such natural fibres might be for example hemp, flax, nettle, cotton, jute, ramie, sisal, and/or any other.

In various instances, the fibres are hemp or flax.

The substrate 8 of the enzyme 4 is able to diffuse within the fibre, in order to reach the preparation 300 able to produce biopesticide and/or repellent for controlling plant pathogens where the substrate 8 will be processed by the active enzyme 4.

Those fibres 200 are further coated by a protective layer, which is a hydrophobic layer 12 in order to be protected from drying.

Such hydrophobic layer is also configured to be permeable to the biopesticide and/or repellent product 10.

The hydrophobic layer 12 can be (3-aminopropyl)triethoxysilane, (3-mercaptopropyl)triethoxysilane, succinic anhydride, alkylketene dimer, 3-isopropenyl-α-α-dimethylbenzyl isocyanate, m-phenylene bismaleimide, vinyl trialkoxysilane, 3-metacryloyloxy propyl trimetoxysilane and/or any other.

The moiety 22, resulting from the cleavage of the precursor 8, stays therefore within the preparation 300 able to produce biopesticide and/or repellent and does not therefore pollute the environment.

As depicted on FIG. 1, the pest protective apparatus 100 of the present invention can further comprise means for distributing water to the at least one fibrous network and/or the at least one fibrous cover 14, the means in various instances comprising a cartridge 16. The cartridge 16 is configured for being in fluid connection with the at least one fibrous network and/or the at least one fibrous cover 14 and for being fed with water. The cartridge 16 can comprise at least one inlet 18 and at least one outlet 20.

Depending whether the pest protective apparatus 100 comprises or does not comprise the cartridge 16, the apparatus 100 is configured either to transform at least one precursor 8, which is incorporated to the preparation 300 able to produce biopesticide and/or repellent by diffusion through the fibre 200, into a biopesticide and/or a repellent product 10; or to incorporate the at least one precursor 8 into the cartridge 16.

Depending whether the pest protective apparatus 100 comprises or does not comprise the cartridge 16, the at least one mineral and/or the at least one cofactor indispensable for the enzyme activity are incorporated either directly within the hydrophilic gel 6 of the preparation 300 able to produce biopesticide and/or repellent; or within the cartridge 16.

The enzymatic reaction is triggered by the addition of water, which plays the role of the solvent of the reaction, bringing subsequently the substrate 8 into contact with the fibres 200 of the preparation 300 able to produce biopesticide and/or repellent, and more particularly, into contact with the active site of the enzyme.

The second utility of water is to bring to the enzyme the at least one mineral and/or the at least one cofactor indispensable for its activity.

Water can come from the rain and/or from artificial means, such for example a drain pump, a hosepipe and/or an irrigation system, and can be channelled to contact the preparation 300 able to produce biopesticide and/or repellent for controlling plant pathogens, triggering subsequently the enzymatic reaction.

When the pest protective apparatus 100 comprises a cartridge 16, the flux of water reaches the cartridge 16 through the inlet 18 which is provided on the cartridge 16.

Once the enzymatic reaction is over, the product 10, which presents biopesticide and/or repellent properties, is released. Generally, the product 10 is a volatile organic compound.

This product 10 acts to eliminate (in case of biopesticide activity) and/or to keep away (in case of repellent activity) the pests and/or the plant pathogens from the living plants, such as bacteria, fungi, insects, bugs, and/or nematodes.

The biopesticide and/or repellent product 10 resulting from the activation of the precursor 8 by addition of water to the pest protective apparatus 100 taught in the present invention is applied to plants, in various instances vegetables and/or fruits, in order to prevent pest attacks.

1-26. (canceled)

27. A pest protective apparatus for controlling plant pathogens, said apparatus comprising at least one of at least one fibrous network and at least one fibrous cover, wherein the at least one of at least one fibrous network and the at least one fibrous cover comprises a system in which at least one precursor is transformed into at least one of a biopesticide and a repellent product.

28. The pest protective apparatus according to claim 27, wherein the system is composed of fibres adapted to control plant pathogens that are coated with at least one preparation able to produce at least one of a biopesticide and a repellent for controlling plant pathogens.

29. The pest protective apparatus according to claim 28, wherein the preparation able to produce the at least one of the biopesticide and the repellent for controlling plant pathogens comprises at least one active enzyme.

30. The pest protective apparatus according to claim 29, wherein the at least one active enzyme is immobilized within one nanoporous material.

31. The pest protective apparatus according to claim 30, wherein the one nanoporous material is a mesoporous material.

32. The pest protective apparatus according to claim 30, wherein the nanoporous material is embedded within one hydrophilic gel.

33. The pest protective apparatus according to claim 32, wherein the hydrophilic gel is made of at least one of alginate, pectin, starch and gelatine.

34. The pest protective apparatus according to claim 29, wherein the at least one active enzyme is one active enzyme selected from at least one of the group of glycosidase, lyase and lachrymatory-factor synthase.

35. The pest protective apparatus according to claim 34, wherein the at least one enzyme from the group of glycosidase is thioglucosidase.

36. The pest protective apparatus according to claim 29, wherein the at least one active enzyme immobilized within the nanoporous material is one of covalently bound to the nanoporous material, physically adsorbed to the nanoporous material, encapsulated within the nanoporous material and entrapped within the nanoporous material.

37. The pest protective apparatus according to claim 36, wherein the at least one active enzyme immobilized within the nanoporous material is cross-linked with glutaraldehyde or with 1-ethyl-3-(3-dimethylaminopropyl)carbodiimide and N-hydroxysuccinimide.

38. The pest protective apparatus according to claim 30, wherein the nanoporous material is a silica-based mesoporous material.

39. The pest protective apparatus according to claim 38, wherein the silica-based mesoporous material comprises at least one of tetraethyl orthosilicate and/or tetramethyl orthosilicate and/or methyltrimethoxysilane and (3-aminopropyl)trimethoxysilane.

40. The pest protective apparatus according to claim 28, wherein the preparation has a pH comprised between 4 and 11.

41. The pest protective apparatus according to claim 28, wherein the preparation further comprises at least one of the at least one precursor, at least one mineral and at least one cofactor.

42. The pest protective apparatus according to claim 41, wherein at least one of the at least one mineral and the at least one cofactor is one of a phosphate buffer and an ascorbic acid.

43. A method for controlling plant pathogens with a pest protective apparatus, wherein the apparatus comprises at least one of at least one fibrous network and at least one fibrous cover, wherein the at least one of at least one fibrous network and the at least one fibrous cover comprises a system in which at least one precursor is transformed into at least one of a biopesticide and a repellent product, said method comprising the following steps:
(a) activating at least one precursor by addition of water to the system of the at least one of the at least one fibrous network and the at least one fibrous cover to produce the at least one of a biopesticide and repellent product for controlling plant pathogens;
(b) applying the at least one of the biopesticide and the repellent product resulting from step (a) to plants in order to prevent pest attacks.

44. The method for controlling plant pathogens according to claim 43, wherein the at least one precursor is comprised in the pest protective apparatus.

45. The method for controlling plant pathogens according to claim 43, wherein the at least one precursor is added to the pest protective apparatus.

46. The method for controlling plant pathogens according to claim 43, wherein the at least one precursor is added with also at least one of at least one mineral and at least one cofactor to the pest protective apparatus.
